(19) United States
(12) Patent Application Publication
(10) Pub. No.: US 2012/0123272 A1
(43) Pub. Date: May 17, 2012

(54) ROTARY ULTRASOUND IMAGING SYSTEM

(76) Inventors: Kwok Ho LAM, Hong Kong (HK); Yan CHEN, Hong Kong (HK); Ji Yan DAI, Hong Kong (HK); Helen L.W. CHAN, Hong Kong (HK)

(21) Appl. No.: 13/221,294

(22) Filed: Aug. 30, 2011

(30) Foreign Application Priority Data
Nov. 12, 2010 (CN) 201010542689.2

(51) Int. Cl. A61B 8/14

(52) U.S. Cl. 600/459

(57) ABSTRACT

This invention relates to a rotary ultrasound imaging system comprising a control device, an ultrasound probe head and a rotary motor device, wherein the rotary motor device receives ultrasound signals sent from the ultrasound probe head, and outputs the received ultrasound signals to the control device through a 360-degree rotation; and the ultrasound probe head comprises a housing with an installation groove which is provided therein with an ultrasound transducer with a concave focusing surface. Since the concave focusing surface is directly formed on the ultrasound transducer, focusing of the rotary ultrasound imaging system is realized without addition of extra components (e.g. lens). Therefore lateral resolution and performance are improved. Furthermore, since the output shaft of the rotary motor of the rotary ultrasound imaging system is configured as a hollow shaft, ultrasound signals from the ultrasound transducer may be sent to the control device through a path of 360-degree rotation. Therefore, complicated modules are not necessary for position alignment and signal connection for 360 degrees rotary ultrasound transducer. The invention may efficiently shield disturbances arising from electrical noise.

### FIELD OF THE INVENTION

The invention relates to a rotary ultrasound imaging system.

### BACKGROUND OF THE INVENTION

Due to the nature of the conventional plane ultrasound transducers, lateral resolution and sound intensity of the resultant images are rather limited, which is particularly the case for high-resolution imaging applications. Therefore, focusing ultrasound transducers are utilized to improve lateral resolution and performance. Shaping a piezoelectric element or adding a lens is a common way to fabricate the focusing ultrasound transducers. It is reported that addition of an extra lens may lead to signal attenuation and acoustic mismatching. Thus, the transducers with shaped elements possess more advantageous in devices with high-sensitivity. Generally, the piezoelectric element is shaped by hard pressing and pressure defection techniques. For the polymers and composite materials, the concave focusing surfaces can be easily fabricated due to their flexibility. However, element degradation and short-circuit may occur since a majority of bulk ceramics or single-crystal elements would be broken apart during hard pressing process.

Endoscopic Ultrasound (EUS) combines endoscopy and ultrasound so as to acquire images and information of the digestive tract or the respiratory system. An endoscopy is typically inserted into the digestive tract via the mouth or the rectum to visualize the surrounding organs or tissues thereof. An ultrasound transducer is directed into the body along with the endoscopy catheter (e.g. gastroscope or intravascular endoscopy) and images the organs or tissues (such as lungs, liver and inner walls of blood vessels) inside the body. As compared to the images obtained by conventional transducers that are placed on the skin directly, the EUS images are more accurate with much detail. This methodology proves to be effective, safe, well tolerated, and minimally-invasive. EUS techniques are mainly based on single-element transducers, especially for intravascular ultrasound imaging, which are mechanically driven by a motor to rotate inside the endoscope to form a 360 degrees scanning image. Even though it is relatively easy to fabricate, its working conditions of the imaging system are often limited by mechanical scanning. Furthermore, a complicated module is required to tackle the alignment of the transducer capable of rotating 360 degrees and to lead out electrical signals.

### BRIEF SUMMARY OF THE INVENTION

The technical problems to be solved by the invention are signal attenuation and acoustic mismatching that may be associated with the prior art rotary ultrasound transducer due to addition of extra lens to achieve focusing, degradation and short-circuit of the ultrasound transducer that may occur due to the hard pressed piezoelectric element, and the necessity of a great number of components, particularly, the component to lead out signal outputs from the ultrasound transducer capable of a 360-degree rotation.

The technical solutions employed in the invention to solve the above-mentioned technical problems is to configure a rotary ultrasound imaging system comprising a control device, an ultrasound probe head and a rotary motor device, wherein the rotary motor device receives ultrasound signals sent from the ultrasound probe head and outputs the received ultrasound signals to the control device through a 360-degree rotation, the ultrasound probe head comprises a housing with an installation groove which is provided therein with an ultrasound transducer with a concave focusing surface.

In the rotary ultrasound imaging system according to the invention, the ultrasound transducer comprises a conductive substrate and a piezoelectric element provided on the top of the conductive substrate. The top of the piezoelectric element is the concave focusing surface with a predetermined radius of curvature. An electrode layer is provided on the concave focusing surface. A matching layer is provided on the electrode layer. Further, the piezoelectric element may be circular or rectangular.

In the rotary ultrasound imaging system according to the invention, the ultrasound transducer and the installation groove is filled with resin materials therebetween.

In the rotary ultrasound imaging system according to the invention, the ultrasound probe head and the rotary motor device are connected with each other via a flexible connector.

In the rotary ultrasound imaging system according to the invention, the rotary motor device comprises a rotary motor with a hollow output shaft through which a connecting cable is interposed, wherein one end of the connecting cable is connected to the ultrasound probe head and the other end thereof is electrically connected to the control device.

In the rotary ultrasound imaging system according to the invention, the connecting cable is a coaxial cable.

In the rotary ultrasound imaging system according to the invention, the flexible connector comprises a flexible metal catheter and a coaxial cable interposed into the flexible metal catheter.

According to a further aspect of the invention, it is provided a machining device for machining the concave focusing surface of the ultrasound transducer, which comprises a rotary mechanism, a grinding mechanism and a pre-pressing mechanism, wherein the rotary mechanism is used to drive the piezoelectric element of the ultrasound transducer to rotate horizontally; the grinding mechanism contacts the piezoelectric element with a certain intersection angle so as to grind the contact surface. The pre-pressing mechanism is used to drive the grinding mechanism to move downwards along with reductions in the thickness of the piezoelectric element.

In the machining device according to the invention, the rotary mechanism comprises a base orientated horizontally and a first rotary motor. The piezoelectric element of the ultrasound transducer is fixed on the base, and the piezoelectric element and the base are provided coaxially on the output shaft of the first rotary motor.

The grinding mechanism comprises a second rotary motor and a grinding wheel provided on the output shaft of the second rotary motor. The grinding wheel contacts the piezoelectric element with a certain intersection angle so as to grind the contact surface between the grinding mechanism and the piezoelectric element.

In the machining device according to the invention, the pre-pressing mechanism is connected to the second rotary motor so as to drive the second rotary motor to move downwards, and thus to drive the grinding wheel to move downwards along with reductions in the thickness of the piezoelectric element.

The rotary ultrasound imaging system possesses many advantages. Since the concave focusing surface is directly formed on the ultrasound transducer, focusing of the rotary ultrasound imaging system is realized without addition of extra components (e.g. lens). Therefore, lateral resolution and performance are improved. Since the output shaft of the rotary motor of the rotary ultrasound imaging system is configured as a hollow shaft, ultrasound signals from the ultrasound transducer may be sent to the control device through a path of 360-degree rotation. Therefore, complicated modules are not necessary for position alignment and signal connection for 360 degrees rotary ultrasound transducer. Furthermore, since the machining device for grinding the concave focusing surface of the piezoelectric element is appropriately configured, the piezoelectric element with a concave focusing surface of a desired radius of curvature may be fabricated without affecting the integrality of ceramics or single-crystal elements. Summarily, the rotary ultrasound imaging system according to the invention is easy to fabricate due to its simpler structure.

### BRIEF DESCRIPTION OF THE DRAWINGS

Hereinafter the invention will be further described with reference to the accompanying figures and embodiments. In the figures,

FIG. 1 is the structural illustration showing the rotary ultrasound imaging system according to the invention;

FIG. 2 is the structural illustration showing the ultrasound probe head in FIG. 1;

FIG. 3 is the cross-sectional view of the section indicated by the dotted line in FIG. 2;

FIG. 4 is the structural illustration showing the rotary motor in FIG. 3;

FIG. 5 is the structural illustration showing the situation in FIG. 4 which the hollow output shaft is installed on the rotary motor;

FIG. 6 is the cross-sectional view of the section indicated by the dotted line in FIG. 5;

FIG. 7 is the structural illustration showing the machining device for the concave focusing surface of the ultrasound transducer according to the invention.

### DETAILED DESCRIPTION OF THE INVENTION

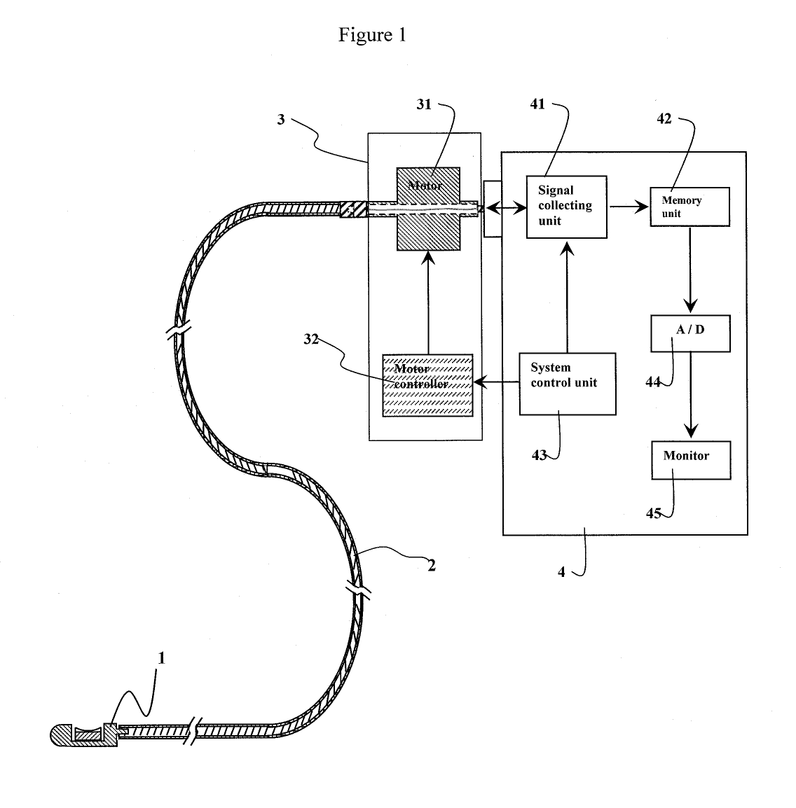

As shown in FIG. 1, the rotary ultrasound imaging system according to the invention mainly comprises three parts, i.e., a control device 4, an ultrasound probe head 1 and a rotary motor device 3. As the part to detect signals, the ultrasound probe head 1 can be introduced into the body or arteries to image the organs or tissues (such as lungs, liver and inner walls of blood vessels) inside the body and to output ultrasound detection signals. The rotary motor device 3 is connected to the ultrasound probe head 1 via a flexible connector 2. Besides mechanically connecting the rotary motor device 3 and the ultrasound probe head 1, the flexible connector 2 also functions to send to the control device 3 the ultrasound detection signals output from the ultrasound probe head 1. During the process that the flexible connector 2 sends ultrasound detection signals to the control device 3, the rotary motor device 3 drives the flexible connector 2 to rotate 360 degrees so as to form a signal output path with a 360-degree rotation.

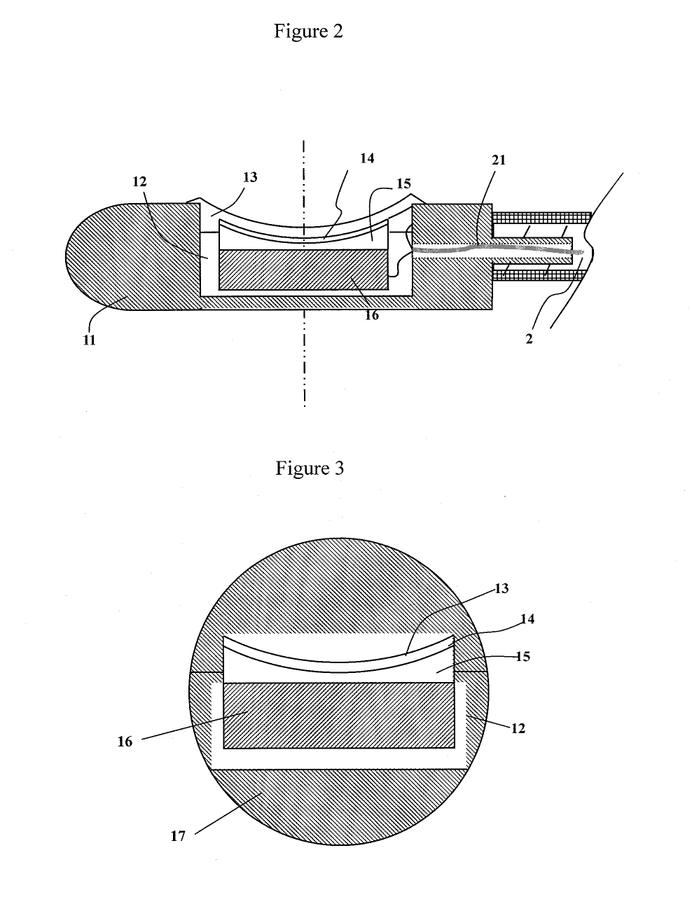

As shown in FIGS. 2 and 3, the ultrasound probe head 1 mainly comprises two parts, i.e., a housing 11 and an ultrasound transducer. The housing 11 is formed with an installation groove in which the ultrasound transducer is placed. For the ultrasound transducer to be fixed in place in the installation groove, the ultrasound transducer and the installation groove is filled with resin material 12 therebetween so as to locate the ultrasound transducer. In the embodiment shown in FIGS. 2 and 3, the ultrasound transducer mainly comprises a conductive substrate 16 and a piezoelectric element 15. On the one hand, the conductive substrate 16 functions as a backing layer to carry the ultrasound transducer. On the other hand it is a transferring device for the electrical signals. The piezoelectric element 15 is located on the top of the conductive substrate 16. A concave focusing surface with a predefined radius of curvature is formed on the top of the piezoelectric element 15 through a dimpling technique. The concave focusing surface is provided thereon with an electrode layer 14 of the same radius of curvature, and the electrode layer 14 is provided thereon with a matching layer 13 also of the same radius of curvature. The piezoelectric element 15 may be circular or rectangular. The matching layer 13 is uniform in structure, and is sandwiched between two media with different sound impedances so as to realize transition or matching of sound impedance. Meanwhile the matching layer 13 provides protection for the piezoelectric element. The matching layer 13 may be made from composite materials consisting of: (1) at least one of Poly-p-xylene polymers and epoxy resin; and (2) at least one of tungsten, tungsten oxide, alumina, titania, silicon oxide, and talc, etc. As shown in FIG. 2, the flexible connector 2 comprises a flexible metal catheter and a coaxial cable 21 interposed into the flexible metal catheter. The conductive wire of the coaxial cable 21 is connected to the conductive substrate 16 such that electronic signals may be routed through the electrode layer 14, the piezoelectric element 15, the conductive substrate 16 and the coaxial cable 21, in this order.

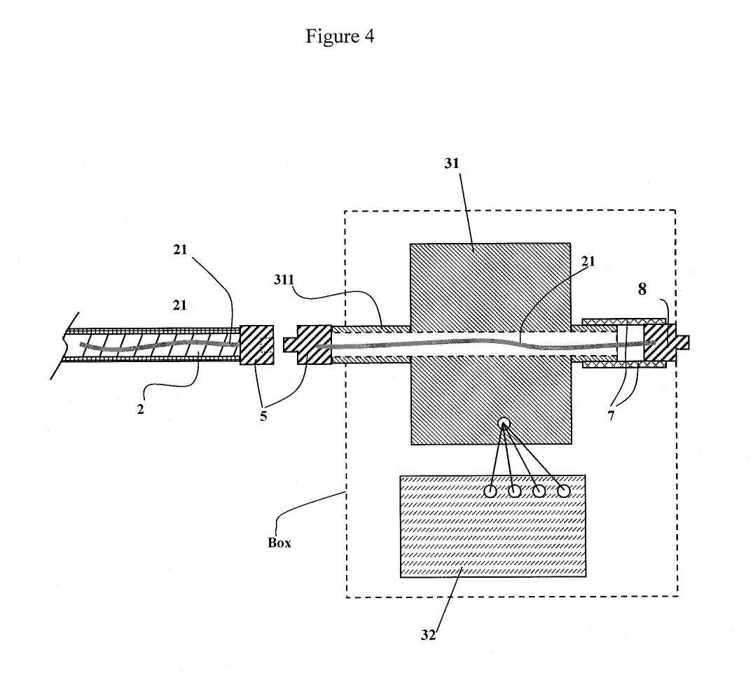

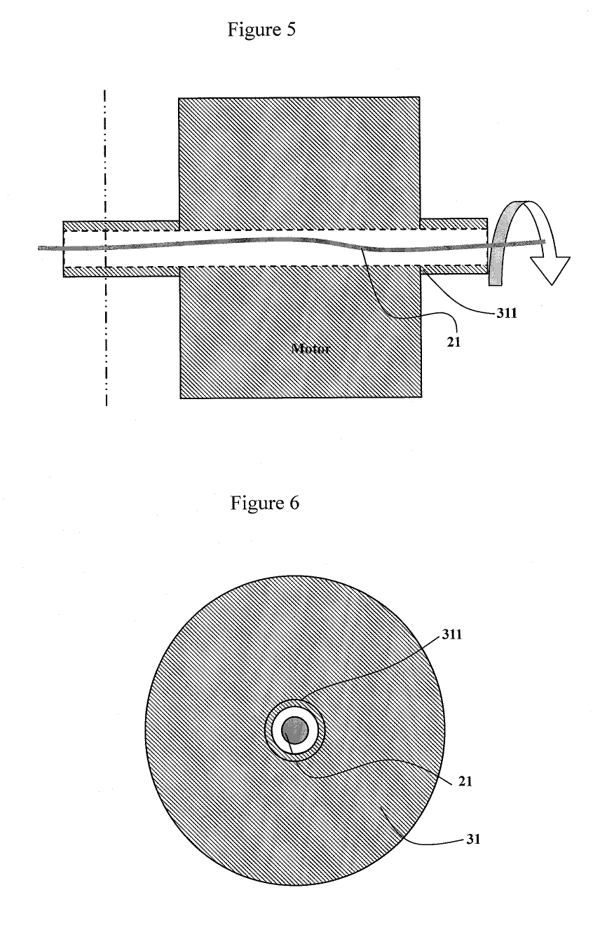

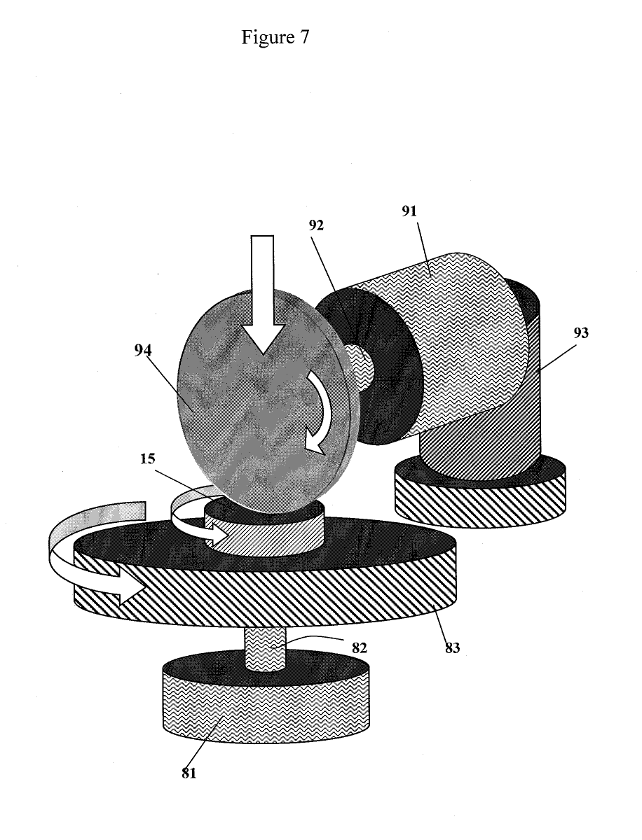

As illustrated in FIG. 4, the rotary motor device 3 mainly comprises a motor controller 32 and a rotary motor 31. The motor controller 32 is electrically connected to the rotary motor 31 so as to control the rotary motor 31 to start or stop rotating and to regulate the rotation frequency thereof. As shown in FIG. 1, the output shaft of the rotary motor 31 is a hollow output shaft 311, one end of which is connected to the flexible connector 2 and the other end of which is connected to the control device 4. The coaxial cable 21 of the flexible connector 2 passes through the hollow shaft 311 so as to be connected with the control device 4 directly. Since the coaxial cable 21 passes through the hollow output shaft 311, the ultrasound probe head 1 may be driven to perform a 360-degree rotation by the rotary motor 31 during the process of sending signals to the control device 4. In this way, signals may be output with a 360-degree rotation. As shown in FIGS. 1 and 4, the hollow output shaft 311 is connected to the flexible connector 2 at one end thereof via a plug device 5, and is connected to the control device 4 at the other end thereof via a plug device 8. The plug device 5 and the plug device 8 are plug devices of the same type. That is, the plug device is comprised of a male plug member and a female socket member. In the embodiment as illustrated in FIG. 4, each end of the hollow output shaft 311 is provided with a male plug member; and the flexible connector 2 and the control device 4 are each provided with a female socket member, respectively. Thus, the hollow output shaft 311 may be connected to the flexible connector 2 and the control device 4 via engagement of the male plug member with the female socket member.

As shown in FIG. 1, the control device 4 comprises a signal collecting unit 41, a memory unit 42, a system control unit 43, an A/D conversion unit 44 and a monitor 45. The signal collecting unit 41, which is connected to the coaxial cable 21, functions to receive ultrasound detection signals transmitted by the coaxial cable 21 during its 360-degrees rotation. The system control unit 43 may function to control the signal collecting unit 41 to collect signals on the one hand, and on the other hand may function to control the motor controller 32 to drive the rotary motor 31. Ultrasound detection signals collected by the signal collecting unit 41 are sent to the memory unit 42 and are then stored therein. These signals are sent to the A/D conversion unit 44 to be converted to digital form, and then sent to the monitor 45 for displaying the detection results from the ultrasound probe head 1.

A machining device may be utilized to grind the concave focusing surface of the piezoelectric element 15 of the ultrasound transducer. The machining device is mainly comprises three parts, i.e. a rotary mechanism, a grinding mechanism and a pre-pressing mechanism, wherein the rotary mechanism functions to drive the piezoelectric element 15 of the ultrasound transducer to rotate horizontally, and the grinding mechanism contacts with the piezoelectric element 15 with a certain intersection angle so as to grind the contact surface therebetween. The grinding mechanism is substantially perpendicular to the piezoelectric element 15; and the rotation of the piezoelectric element 15 functions to grind the contact surface evenly. The pre-pressing mechanism moves downwards with thickness of the piezoelectric element decreasing so as to grind continuously.

In the embodiment shown in FIG. 7, the rotary mechanism comprises a base 83 orientated horizontally and a first rotary motor 81. The piezoelectric element 15 of the ultrasound transducer is fixed on the base 83, and the piezoelectric element 15 and the base 83 are coaxially provided on the output shaft 82 of the first rotary motor 81. Thus the base 83 and the piezoelectric element 15 may be driven by the first rotary motor 81 to rotate horizontally. The grinding mechanism comprises a second rotary motor 91 and a grinding wheel 94 provided on the output shaft 92 of the second rotary motor 91. The grinding wheel 94 contacts the piezoelectric element 15 with a certain intersection angle so as to grind the contact surface between the grinding mechanism and the piezoelectric element. In the embodiment, the grinding wheel 94 is orthogonal to the piezoelectric element 15. The pre-pressing mechanism 93 is elastically engaged with the second rotary motor 91 so as to drive the second rotary motor 91 to move downwards. In this way the grinding wheel 94 is driven to move downwards so as to grind continually. The pre-pressing mechanism 93 may be designed in a conventional way in which the downward pressure is regulated through adjusting the distance measured from the center of gravity of the grinding wheel 94 to the sample.

Although the invention has been described through illustrating particular embodiments, the skilled in the art should appreciate that embodiments and the features thereof may be altered or equivalently substituted without departing from the spirit and scope of the present invention. Furthermore, the embodiments and the features thereof may be modified so as to match the specific applications and materials without departing from the spirit and the scope of the invention. Consequently, the present invention is in no way limited to the embodiments disclosed therein, and it is intended that all embodiments falling into the scope of claims of the application are within the protection scope claimed by the invention.

1. A rotary ultrasound imaging system, comprising:
a control device;
an ultrasound probe head; and
a rotary motor device;
wherein the rotary motor device receives ultrasound signals sent from the ultrasound probe head, and the rotary motor device outputs the received ultrasound signals to the control device through a 360-degree rotation; and
the ultrasound probe head comprises a housing with an installation groove which is provided therein with an ultrasound transducer with a concave focusing surface.

2. The rotary ultrasound imaging system according to claim 1, wherein the ultrasound transducer comprises a conductive substrate as the backing layer and a piezoelectric element provided on the top the conductive substrate; the top of the piezoelectric element is the concave focusing surface with a predetermined radius of curvature; an electrode layer is provided on the concave focusing surface, and a matching layer is provided on the electrode layer; the piezoelectric element is circular or rectangular.

3. The rotary ultrasound imaging system according to claim 1, wherein the ultrasound transducer and the installation groove is filled with resin materials therebetween.

4. The rotary ultrasound imaging system according to claim 1, wherein the ultrasound probe head and the rotary motor device are connected with each other via a flexible connector.

5. The rotary ultrasound imaging system according to claim 1, wherein the rotary motor device comprises a rotary motor with a hollow output shaft through which a connecting cable is interposed, and one end of the connecting cable is connected to the ultrasound probe head and the other end thereof is electrically connected to the control device.

6. The rotary ultrasound imaging system according to claim 5, wherein the connecting cable is a coaxial cable.

7. The rotary ultrasound imaging system according to claim 4, wherein the flexible connector comprises a flexible metal catheter and a coaxial cable interposed into the flexible metal catheter.

8. A machining device for machining the concave focusing surface of the ultrasound transducer according to claims 1 to 4, comprising:
a rotary mechanism;
a grinding mechanism; and
a pre-pressing mechanism;
wherein the rotary mechanism is used to drive a piezoelectric element of the ultrasound transducer to rotate horizontally;
the grinding mechanism contacts the piezoelectric element with a certain intersection angle so as to grind a contact surface between the grinding mechanism and the piezoelectric element; and
the pre-pressing mechanism is used to drive the grinding mechanism to move downwards along with reductions in the thickness of the piezoelectric element.

9. The machining device according to claim 8, wherein the rotary mechanism comprises a base orientated horizontally and a first rotary motor, the piezoelectric element of the ultrasound transducer is fixed on the base, and the piezoelectric element and the base are provided coaxially on the output shaft of the first rotary motor; and
the grinding mechanism comprises a second rotary motor and a grinding wheel provided on the output shaft of the second rotary motor; and the grinding wheel contacts the piezoelectric element with a certain intersection angle so as to grind the contact surface between the grinding mechanism and the piezoelectric element.

10. The machining device according to claim 9, wherein the pre-pressing mechanism is connected to the second rotary motor so as to drive the second rotary motor to move downwards, which drives the grinding wheel to move downwards along with reductions in the thickness of the piezoelectric element.
